(19) United States
(12) Patent Application Publication
(10) Pub. No.: US 2026/0041558 A1
(43) Pub. Date: Feb. 12, 2026

(54) BIOENGINEERING PATIENT-SPECIFIC 3D-PRINTED CARTILAGE IMPLANTS

(71) Applicants: Sheba Impact Ltd., Ramat-Gan (IL); Technion Research & Development Foundation Limited, Haifa (IL)

(72) Inventors: Shay DUVDEVANI, Ramat Gan (IL); Orit HARARI-STEINBERG, RaAnana (IL); Or BENIFLA, Tel Aviv (IL); Shulamit LEVENBERG, Haifa (IL)

(73) Assignees: Sheba Impact Ltd., Ramat-Gan (IL); Technion Research & Development Foundation Limited, Haifa (IL)

(21) Appl. No.: 19/100,610

(22) PCT Filed: Aug. 3, 2023
(86) PCT No.: PCT/IL2023/050813

§ 371 Date: Feb. 3, 2025

(30) Foreign Application Priority Data
Aug. 3, 2022 (IL) 295342

(51) Int. Cl. A61F 2/30; A61F 2/18; A61L 27/28

(52) U.S. Cl. CPC A61F 2/30756; A61F 2/186; A61F 2/3094; A61L 27/28; A61F 2002/30011; A61F 2002/30242; A61F 2002/30677; A61F 2002/30761; A61F 2002/30766; A61F 2002/30985; A61L 2400/18

(57) ABSTRACT

The invention related to an implant comprising a scaffold comprising at least one first area characterized by micropores and at least one second area characterized by macropores, wherein said at least one second area is defined by being expected to be exposed to higher pressures and/or forces when compared with said at least one first area, and methods thereof.

### RELATED APPLICATION/S

This application claims the benefit of priority of Israeli Patent Application No. 295342 filed on 3 Aug. 2022, the contents of which are incorporated herein by reference in their entirety.

### FIELD AND BACKGROUND OF THE INVENTION

The present invention, in some embodiments thereof, relates to 3D printed cartilage implants and, more particularly, but not exclusively, to 3D printed cartilage implants comprising both spheroids and single cells. Cartilage reconstruction following trauma (e.g., burns, cancer), or congenital anomalies is extremely challenging. Cartilage does not heal itself and the cartilage cells known as chondrocytes do not replicate or repair themselves, making damaged or injured cartilage unlikely to heal well without medical intervention. Current solutions are artificial external prosthesis, which are synthetic implants that are surgically implanted under the skin and autologous cartilage reconstruction, which is a complex surgical procedure forcing harvesting of costal cartilage, are deficient and ineffective, as they can cause morbidity and mortality.

Additional background art includes U.S. Pat. No. 10,532,126B2 disclosing a method of providing a graft scaffold for cartilage repair, particularly in a human patient comprising the steps of providing particles and/or fibres; providing an aqueous solution of a gelling polysaccharide; providing mammalian cells; mixing said particles and/or fibres, said aqueous solution of a gelling polysaccharide and said mammalian cells to obtain a printing mix; and depositing said printing mix in a three-dimensional form. The patent further discloses graft scaffolds and grafts obtained by the method.

U.S. Pat. No. 9,044,335B2 disclosing a tissue-engineered intervertebral disc (IVD) suitable for total disc replacement in a mammal and methods of fabrication. The IVD comprises a nucleus pulposus structure comprising a first population of living cells that secrete a hydrophilic protein and an annulus fibrosis structure surrounding and in contact with the nucleus pulposus structure, the annulus fibrosis structure comprising a second population of living cells and type I collagen. The collagen fibrils in the annulus fibrosis structure are circumferentially aligned around the nucleus pulposus region due to cell-mediated contraction in the annulus fibrosis structure. Also disclosed are methods of fabricating tissue-engineered intervertebral discs.

U.S. Patent Application Publication No. US20220016314A1 disclosing a tissue or organ replacement including a tissue-engineered construct that includes one or more bio ink compositions and a biocompatible support structure. The support structure includes one or more external supports, one or more internal supports, or combinations thereof of a biocompatible material. The composition has a three-dimensional (3D) shape, and the biocompatible material is present in an amount of about 1% to about 100% by weight of the biocompatible support structure.

International Patent Application Publication No. WO2020240040A1 disclosing a method for propagating or enriching cartilage cells and providing spheroids thereof, where the spheroids are useful for an autologous chondrocyte implantation (ACI) product. The patent also discloses the production of spheroids from articular cartilage and use thereof.

Scientific publication “Biofabrication of spatially organised tissues by directing the growth of cellular spheroids within 3D printed polymeric microchambers” by Daly et al, disclosing biofabrication strategies that enable the engineering of structurally organised tissues by guiding the growth of cellular spheroids within arrays of 3D printed polymeric microchambers.

Scientific publication “Influence of pore sizes in 3D-scaffolds on mechanical properties of scaffolds and survival, distribution, and proliferation of human chondrocytes” by Abpeikar et al, disclosing evaluation of the effects of pore size in scaffolds on mechanical properties and chondrocyte-scaffold interactions.

Scientific publication “Translational Application of 3D Bioprinting for Cartilage Tissue Engineering” by McGivern et al, disclosing a review about developments in 3D bioprinting for cartilage tissue engineering. The bioink and construct properties required for successful application in cartilage repair applications are highlighted. Furthermore, the potential for translation of 3D bioprinted constructs to the clinic is discussed.

Scientific publication “3D Bioprinted Implants for Cartilage Repair in Intervertebral Discs and Knee Menisci“ by Perera et al, disclosing a review on advances in 3D bioprinting for cartilage tissue engineering for knee menisci and intervertebral disc repair. Additionally, it is disclosed medical-grade materials and techniques that can be used for printing.

Scientific publication “3D-bioprinting a genetically inspired cartilage scaffold with GDF5-conjugated BMSC-laden hydrogel and polymer for cartilage repair” by Sun et al, disclosing a functional knee articular cartilage construct for cartilage repair by 3d-bioprinting a GDF5-conjugated BMSC-laden scaffold.

### SUMMARY OF THE INVENTION

According to an aspect of some embodiments of the present invention there is provided an implant comprising a scaffold comprising at least one first area characterized by micropores and at least one second area characterized by macropores, wherein said at least one second area is defined by being expected to be exposed to higher pressures and/or forces when compared with said at least one first area.

According to some embodiments of the invention, the implant further comprises at least one second scaffold located below said scaffold, and configured to provide mechanical support to said scaffold.

According to some embodiments of the invention, the implant further comprises at least one bio-ink material configured to allow attachment of a plurality of cells to a surface of said implant.

According to some embodiments of the invention, said bio-ink material is one or more of fibrin, hydrogel and amino-acids.

According to some embodiments of the invention, said bio-ink material is configured to be any one or combination of:
a. applied on a surface of said scaffold;
b. part of materials which said scaffold are made of.

According to some embodiments of the invention, said implant comprises a plurality of spheroids.

According to some embodiments of the invention, said plurality of spheroids are chondro-spheroids.

According to some embodiments of the invention, said macropores are configured in size for housing said plurality of spheroids.

According to some embodiments of the invention, said implant further comprises single cells.

According to some embodiments of the invention, said plurality of spheroids are formed from one or more of expanded chondrocyte cells and mesenchymal stem cells.

According to some embodiments of the invention, said at least one first area and said at least one second area, each comprise different mechanical characteristics from the other.

According to some embodiments of the invention, said micropores are smaller than about 100 microns.

According to some embodiments of the invention, said micropores comprise a size from about 10 microns to about 100 microns.

According to some embodiments of the invention, said macropores comprise one or more of medium size macropores, large size macropores and extra-large macropores.

According to some embodiments of the invention, said medium size macropores comprise a size from about 400 microns to about 600 microns.

According to some embodiments of the invention, said large size macropores comprise a size from about 500 microns to about 900 microns.

According to some embodiments of the invention, said extra-large size macropores comprise a size from about 800 microns to about 1200 microns.

According to some embodiments of the invention, said at least one first area comprises
a. from about 15% to about 30% extra-large pores having a size of from about 0.8 mm to about 1.2 mm;
b. from about 0% to about 30% large pores having a size of from about 0.5 mm to about 0.9 mm; and
c. from about 40% to about 75% medium pores having a size of from about 0.4 mm to about 0.6 mm.

According to some embodiments of the invention, said at least one second area comprises one or more of medium size macropores, large size macropores and extra-large macropores.

According to some embodiments of the invention, said medium size macropores comprise a size from about 400 microns to about 600 microns.

According to some embodiments of the invention, said large size macropores comprise a size from about 500 microns to about 900 microns.

According to some embodiments of the invention, said extra-large size macropores comprise a size from about 800 microns to about 1200 microns.

According to some embodiments of the invention, said at least one second area comprises
a. from about 5% to about 20% extra-large pores having a size of from about 0.8 mm to about 1.2 mm;
b. from about 5% to about 25% large pores having a size of from about 0.5 mm to about 0.9 mm; and
c. from about 55% to about 90% medium pores having a size of from about 0.4 mm to about 0.6 mm.

According to some embodiments of the invention, said at least one second area is configured to withstand higher pressure levels when compared with said at least one first area.

According to some embodiments of the invention, said scaffold is characterized by at least one first area comprising one or more of micropores and macropores; and at least one second area comprising macropores; said at least one second area configured to withstand higher pressures and/or forces when compared with said at least one first area.

According to some embodiments of the invention, said at least one second scaffold comprises micropores.

According to some embodiments of the invention, said micropores are smaller than about 100 microns.

According to some embodiments of the invention, said micropores comprise a size from about 10 microns to about 100 microns.

According to some embodiments of the invention, said micropores comprise a size from about 400 microns to about 600 microns.

According to some embodiments of the invention, said implant comprises a form of an external surface of a location where said implant is needed to be implanted.

According to some embodiments of the invention, said implant comprises a nose-shape form.

According to some embodiments of the invention, said at least one second scaffold comprises a form of an internal surface of a location where said implant is needed to be implanted.

According to some embodiments of the invention, said at least one second scaffold comprises a nasal-canal form.

According to some embodiments of the invention, said scaffold is configured to degrade about 3 months after implantation.

According to some embodiments of the invention, said at least one second scaffold is configured to degrade about 6 months after implantation.

According to some embodiments of the invention, said scaffold and said at least one second scaffold are configured to degrade at different time windows.

According to some embodiments of the invention, said at least one second scaffold is configured to degrade after said scaffold.

According to some embodiments of the invention, said scaffold is configured to degrade before said at least one second scaffold.

According to some embodiments of the invention, said scaffold and said at least one second scaffold are made of a biodegradable polymer material.

According to some embodiments of the invention, said biodegradable polymer material is polydioxanone.

According to some embodiments of the invention, said scaffold and said at least one second scaffold are made of different materials.

According to some embodiments of the invention, said implant is adapted to be implanted in one or more locations on a patient comprising one or more of nose, larynx, ribs, trachea, external ear, any kind of bone and joints.

According to some embodiments of the invention, said implant is configured for knee form.

According to some embodiments of the invention, said implant is configured for joints form.

According to some embodiments of the invention, the implant is configured for the reconstruction of cartilage in one or more of the knee, the tibia, the femur and the patella. For example, reconstruction of any articular cartilage due to injury or defect.

According to some embodiments of the invention, the implant further comprises one or more of drugs, antibiotics, steroids and anticoagulants configured to be released from said implant after implantation.

According to an aspect of some embodiments of the present invention there is provided a method of manufacturing an implant, comprising printing, optionally direct 3D printing, a scaffold comprising at least one first area characterized by micropores and at least one second area characterized by macropores.

According to some embodiments of the invention, the method further comprises defining said at least one second area by assessing areas which are expected to be exposed to higher pressures and/or forces when compared with said at least one first area.

According to some embodiments of the invention, the method further comprises configuring said macropores so as to allow seeding spheroids therein.

According to some embodiments of the invention, the method further comprises seeding said spheroids on said scaffold.

According to some embodiments of the invention, the method further comprises seeding single cells on said scaffold.

According to some embodiments of the invention, the method further comprises printing at least one second scaffold, optionally direct 3D printing, said printing said at least one second scaffold comprising providing said at least one second scaffold with a form so as to provide mechanical support to said scaffold.

According to some embodiments of the invention, the method further comprises providing mechanical support to said scaffold by positioning said at least one second scaffold below said scaffold.

According to some embodiments of the invention, the method further comprises adding at least one bio-ink material to said implant, said bio-ink material being configured to allow attachment of a plurality of cells to a surface of said implant.

According to some embodiments of the invention, said bio-ink material is one or more of fibrin, hydrogel and amino-acids.

According to some embodiments of the invention, said adding at least one bio-ink material comprises one or more of:
a. applying said bio-ink on a surface of said scaffold; and
b. adding said bio-ink material to materials which said scaffold are made of.

According to some embodiments of the invention, said macropores comprise a size smaller than about 100 microns.

According to some embodiments of the invention, said macropores comprise a size from about 10 microns to about 100 microns.

According to some embodiments of the invention, said macropores comprise one or more of medium size macropores, large size macropores and extra-large macropores.

According to some embodiments of the invention, said medium size macropores comprise a size from about 400 microns to about 600 microns.

According to some embodiments of the invention, said large size macropores comprise a size from about 500 microns to about 900 microns.

According to some embodiments of the invention, said extra-large size macropores comprise a size from about 800 microns to about 1200 microns.

According to some embodiments of the invention, said printing said scaffold comprises printing said scaffold with at least one first area and at least one second area.

According to some embodiments of the invention, said at least one first area comprises
a. from about 15% to about 30% extra-large pores having a size of from about 0.8 mm to about 1.2 mm;
b. from about 0% to about 30% large pores having a size of from about 0.5 mm to about 0.9 mm; and
c. from about 40% to about 75% medium pores having a size of from about 0.4 mm to about 0.6 mm.

According to some embodiments of the invention, said at least one second area comprises one or more of medium size macropores, large size macropores and extra-large macropores.

According to some embodiments of the invention, said medium size macropores comprise a size from about 400 microns to about 600 microns.

According to some embodiments of the invention, said large size macropores comprise a size from about 500 microns to about 900 microns.

According to some embodiments of the invention, said extra-large size macropores comprise a size from about 800 microns to about 1200 microns.

According to some embodiments of the invention, said at least one second area comprises
a. from about 5% to about 20% extra-large pores having a size of from about 0.8 mm to about 1.2 mm;
b. from about 5% to about 25% large pores having a size of from about 0.5 mm to about 0.9 mm; and
c. from about 55% to about 90% medium pores having a size of from about 0.4 mm to about 0.6 mm.

According to some embodiments of the invention, said at least one second area is configured to withstand higher pressure levels when compared with said at least one first area.

According to some embodiments of the invention, said scaffold is characterized by at least one first area comprising one or more of micropores and macropores; and at least one second area comprising macropores; said at least one second area configured to withstand higher pressures and/or forces when compared with said at least one first area.

According to some embodiments of the invention, said at least one second area is defined by being expected to be exposed to higher pressures and/or forces when compared with said at least one first area.

According to some embodiments of the invention, said printing said at least one second scaffold comprises printing said at least one second with micropores.

According to some embodiments of the invention, said micropores are smaller than about 100 microns.

According to some embodiments of the invention, said micropores comprise a size from about 10 microns to about 100 microns.

According to some embodiments of the invention, said micropores comprise a size from about 400 microns to about 600 microns.

According to some embodiments of the invention, said printing said scaffold comprises printing said scaffold with a form of an external surface of a location where said implant is needed to be implanted.

According to some embodiments of the invention, said printing said scaffold comprises printing said scaffold with a nose-shape form.

According to some embodiments of the invention, said printing said at least one second scaffold comprises printing said at least one second scaffold with a form of an internal surface of a location where said implant is needed to be implanted.

According to some embodiments of the invention, said printing said at least one second scaffold comprises printing said at least one second scaffold with a nasal-canal form.

According to some embodiments of the invention, said scaffold is configured to degrade about 3 months after implantation.

According to some embodiments of the invention, said at least one second scaffold is configured to degrade about 6 months after implantation.

According to some embodiments of the invention, said first scaffold and said at least one second scaffold are configured to degrade at different time windows.

According to some embodiments of the invention, said at least one second scaffold is configured to degrade after said first scaffold.

According to some embodiments of the invention, said first scaffold is configured to degrade before said at least one second scaffold.

According to some embodiments of the invention, first scaffold and said at least one second scaffold are made of a biodegradable polymer material.

According to some embodiments of the invention, said biodegradable polymer material is polydioxanone.

According to some embodiments of the invention, said first scaffold and said at least one second scaffold are made of different materials.

According to some embodiments of the invention, said implant is adapted to be implanted in one or more locations on a patient comprising one or more of nose, larynx, ribs, trachea, external ear and joints.

According to some embodiments of the invention, said spheroids are chondro-spheroids.

According to some embodiments of the invention, said spheroids are formed from one or more of expanded chondrocyte cells and mesenchymal stem cells.

According to an aspect of some embodiments of the present invention there is provided an implant, comprising:
a. a first scaffold comprising a plurality of spheroids;
b. at least one second scaffold located below said first scaffold, and configured to provide mechanical support to said first scaffold.

According to some embodiments of the invention, said first scaffold is characterized by at least one first area and at least one second area, each comprising different mechanical characteristics from the other.

According to some embodiments of the invention, said at least one first area comprises one or more of micropores and macropores.

According to some embodiments of the invention, said micropores are smaller than about microns.

According to some embodiments of the invention, said micropores comprise a size from about 10 microns to about 100 microns.

According to some embodiments of the invention, said macropores comprise one or more of medium size macropores, large size macropores and extra-large macropores.

According to some embodiments of the invention, said medium size macropores comprise a size from about 400 microns to about 600 microns.

According to some embodiments of the invention, said large size macropores comprise a size from about 500 microns to about 900 microns.

According to some embodiments of the invention, said extra-large size macropores comprise a size from about 800 microns to about 1200 microns.

According to some embodiments of the invention, said at least one first area comprises
a. from about 15% to about 30% extra-large pores having a size of from about 0.8 mm to about 1.2 mm;
b. from about 0% to about 30% large pores having a size of from about 0.5 mm to about 0.9 mm; and
c. from about 40% to about 75% medium pores having a size of from about 0.4 mm to about 0.6 mm.

According to some embodiments of the invention, said at least one second area comprises one or more of medium size macropores, large size macropores and extra-large macropores.

According to some embodiments of the invention, said medium size macropores comprise a size from about 400 microns to about 600 microns.

According to some embodiments of the invention, said large size macropores comprise a size from about 500 microns to about 900 microns.

According to some embodiments of the invention, said extra-large size macropores comprise a size from about 800 microns to about 1200 microns.

According to some embodiments of the invention, said at least one second area comprises
a. from about 5% to about 20% extra-large pores having a size of from about 0.8 mm to about 1.2 mm;
b. from about 5% to about 25% large pores having a size of from about 0.5 mm to about 0.9 mm; and
c. from about 55% to about 90% medium pores having a size of from about 0.4 mm to about 0.6 mm.

According to some embodiments of the invention, said at least one second area is configured to withstand higher pressure levels when compared with said at least one first area.

According to some embodiments of the invention, said first scaffold is characterized by at least one first area comprising one or more of micropores and macropores; and at least one second area comprising macropores; said at least one second area configured to withstand higher pressures and/or forces when compared with said at least one first area.

According to some embodiments of the invention, at least one second area is defined by being expected to be exposed to higher pressures and/or forces when compared with said at least one first area.

According to some embodiments of the invention, said at least one second scaffold comprises micropores.

According to some embodiments of the invention, said micropores are smaller than about 100 microns.

According to some embodiments of the invention, said micropores comprise a size from about 10 microns to about 100 microns.

According to some embodiments of the invention, said micropores comprise a size from about 400 microns to about 600 microns.

According to some embodiments of the invention, said first scaffold comprises a form of an external surface of a location where said implant is needed to be implanted.

According to some embodiments of the invention, said first scaffold comprises a nose-shape form.

According to some embodiments of the invention, said at least one second scaffold comprises a form of an internal surface of a location where said implant is needed to be implanted.

According to some embodiments of the invention, said at least one second scaffold comprises a nasal-canal form.

According to some embodiments of the invention, said first scaffold is configured to degrade about 3 months after implantation.

According to some embodiments of the invention, said at least one second scaffold is configured to degrade about 6 months after implantation.

According to some embodiments of the invention, said scaffold and said at least one second scaffold are configured to degrade at different time windows.

According to some embodiments of the invention, said at least one second scaffold is configured to degrade after said scaffold.

According to some embodiments of the invention, said scaffold is configured to degrade before said at least one second scaffold.

According to some embodiments of the invention, said first scaffold and said at least one second scaffold are made of a biodegradable polymer material.

According to some embodiments of the invention, said biodegradable polymer material is polydioxanone.

According to some embodiments of the invention, said first scaffold and said at least one second scaffold are made of different materials.

According to some embodiments of the invention, said implant is adapted to be implanted in one or more locations on a patient comprising one or more of nose, larynx, ribs, trachea, external ear and joints.

According to some embodiments of the invention, said plurality of spheroids are chondro-spheroids.

According to some embodiments of the invention, said first scaffold further comprises single cells.

According to some embodiments of the invention, said plurality of spheroids are formed from one or more of expanded chondrocyte cells and mesenchymal stem cells.

According to some embodiments of the invention, the implant further comprises at least one bio-ink material configured to allow attachment of a plurality of cells to a surface of said implant.

According to some embodiments of the invention, said bio-ink material is one or more of fibrin, hydrogel and amino-acids.

According to some embodiments of the invention, said bio-ink material is configured to be:
a. applied on a surface of said scaffold;
b. part of materials which said scaffold are made of;
c. any combination thereof.

According to some embodiments of the invention, the implant further comprises one or more of drugs, antibiotics, steroids and anticoagulants configured to be released from said implant after implantation.

According to an aspect of some embodiments of the present invention there is provided a method of manufacturing an implant, comprising:
a. printing a first scaffold, said printing said first scaffold comprising providing said first scaffold with a plurality of macropores so as to allow seeding spheroids therein;
b. seeding spheroids on said first scaffold;
c. printing at least one second scaffold, said printing said at least one second scaffold comprising providing said at least one second scaffold with a form so as to provide mechanical support to said first scaffold.

According to some embodiments of the invention, said providing said first scaffold with a plurality of macropores comprises providing a plurality of macropores having a size smaller than about 100 microns.

According to some embodiments of the invention, said providing said first scaffold with a plurality of macropores comprises providing a plurality of macropores having a size from about 10 microns to about 100 microns.

According to some embodiments of the invention, said providing said first scaffold with a plurality of macropores comprises providing a plurality of macropores comprising one or more of medium size macropores, large size macropores and extra-large macropores.

According to some embodiments of the invention, said medium size macropores comprise a size from about 400 microns to about 600 microns.

According to some embodiments of the invention, said large size macropores comprise a size from about 500 microns to about 900 microns.

According to some embodiments of the invention, said extra-large size macropores comprise a size from about 800 microns to about 1200 microns.

According to some embodiments of the invention, said printing said first scaffold comprises printing said first scaffold with at least one first area and at least one second area.

According to some embodiments of the invention, said at least one first area comprises
a. from about 15% to about 30% extra-large pores having a size of from about 0.8 mm to about 1.2 mm;
b. from about 0% to about 30% large pores having a size of from about 0.5 mm to about 0.9 mm; and
c. from about 40% to about 75% medium pores having a size of from about 0.4 mm to about 0.6 mm.

According to some embodiments of the invention, said at least one second area comprises one or more of medium size macropores, large size macropores and extra-large macropores.

According to some embodiments of the invention, said medium size macropores comprise a size from about 400 microns to about 600 microns.

According to some embodiments of the invention, said large size macropores comprise a size from about 500 microns to about 900 microns.

According to some embodiments of the invention, said extra-large size macropores comprise a size from about 800 microns to about 1200 microns.

According to some embodiments of the invention, said at least one second area comprises
a. from about 5% to about 20% extra-large pores having a size of from about 0.8 mm to about 1.2 mm;
b. from about 5% to about 25% large pores having a size of from about 0.5 mm to about 0.9 mm; and
c. from about 55% to about 90% medium pores having a size of from about 0.4 mm to about 0.6 mm.

According to some embodiments of the invention, said at least one second area is configured to withstand higher pressure levels when compared with said at least one first area.

According to some embodiments of the invention, said first scaffold is characterized by at least one first area comprising one or more of micropores and macropores; and at least one second area comprising macropores; said at least one second area configured to withstand higher pressures and/or forces when compared with said at least one first area.

According to some embodiments of the invention, said at least one second area is defined by being expected to be exposed to higher pressures and/or forces when compared with said at least one first area.

According to some embodiments of the invention, said printing said at least one second scaffold comprises printing said at least one second with micropores.

According to some embodiments of the invention, said micropores are smaller than about 100 microns.

According to some embodiments of the invention, said micropores comprise a size from about 10 microns to about 100 microns.

According to some embodiments of the invention, said micropores comprise a size from about 400 microns to about 600 microns.

According to some embodiments of the invention, said printing said first scaffold comprises printing said first scaffold with a form of an external surface of a location where said implant is needed to be implanted.

According to some embodiments of the invention, said printing said first scaffold comprises printing said first scaffold with a nose-shape form.

According to some embodiments of the invention, said printing said at least one second scaffold comprises printing said at least one second scaffold with a form of an internal surface of a location where said implant is needed to be implanted.

According to some embodiments of the invention, said printing said at least one second scaffold comprises printing said at least one second scaffold with a nasal-canal form.

According to some embodiments of the invention, said first scaffold is configured to degrade about 3 months after implantation.

According to some embodiments of the invention, said at least one second scaffold is configured to degrade about 6 months after implantation.

According to some embodiments of the invention, said scaffold and said at least one second scaffold are configured to degrade at different time windows.

According to some embodiments of the invention, said at least one second scaffold is configured to degrade after said scaffold.

According to some embodiments of the invention, said scaffold is configured to degrade before said at least one second scaffold.

According to some embodiments of the invention, said first scaffold and said at least one second scaffold are made of a biodegradable polymer material.

According to some embodiments of the invention, said biodegradable polymer material is polydioxanone.

According to some embodiments of the invention, said first scaffold and said at least one second scaffold are made of different materials.

According to some embodiments of the invention, said implant is adapted to be implanted in one or more locations on a patient comprising one or more of nose, larynx, ribs, trachea, external ear and joints.

According to some embodiments of the invention, said spheroids are chondro-spheroids.

According to some embodiments of the invention, said first scaffold further comprises single cells.

According to some embodiments of the invention, said spheroids are formed from one or more of expanded chondrocyte cells and mesenchymal stem cells.

According to some embodiments of the invention, the method further comprises adding one or more of drugs, antibiotics, steroids and anticoagulants configured to be released from said implant after implantation.

According to an aspect of some embodiments of the present invention there is provided a method of treatment for implanting an implant, comprising:
a. generating a first part of said implant comprising an first degradation timeline;
b. generating a second part of said implant comprising an second degradation timeline;
c. assembling said first part with said second part;
d. implanting said implant into a patient.

According to some embodiments of the invention, the method further comprises implanting a plurality of cells into said first part of said implant.

According to some embodiments of the invention, said plurality of cells are a plurality of spheroids.

According to some embodiments of the invention, said assembling said first part of said implant with said second part of said implant comprises assembling so said second part of said implant provides structural support to said first part of said implant.

According to some embodiments of the invention, said first part of said implant and said second part of said implant are both scaffolds.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and/or images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the Drawings:

FIG. 1 is a schematic representation of an exemplary implant, according to some embodiments of the invention;

FIG. 2 is a schematic representation of an exemplary architecture of an exemplary nose-shape layer, according to some embodiments of the invention;

FIG. 3 is a schematic representation of an exemplary process of identification of areas under more mechanical stress and an exemplary design of support scaffold, according to some embodiments of the invention;

FIG. 4a is a flowchart of an exemplary process of preparation of an exemplary implant, according to some embodiments of the invention;

FIG. 4b is a schematic representation of an exemplary timeline of implantation, according to some embodiments of the invention;

FIGS. 5a-b are of light microscope images of the formation of chondro-spheroids, according to some embodiments of the invention;

FIG. 6 is a graph showing the results of cartilage formation in scaffold-free chondrocytes in vitro in 2D cell culture vs. chondro-spheroids, according to some embodiments of the invention;

FIGS. 7a-c are images showing representative microscope images of spheroid differentiation within bio-ink and exemplary matured and functional engineered cartilage tissue, according to some embodiments of the invention;

FIGS. 8a-c are images showing exemplary in vitro maturation of spheroids-based PDO scaffold-based engineered cartilage, according to some embodiments of the invention;

FIGS. 9a-d are images showing an in vitro partial maturation cartilage tissue with typical morphology, an H&E staining, a table showing the results of the collagen and proteoglycan assay and an exemplary in vivo maturation process in nude mice, according to some embodiments of the invention;

FIG. 10 is a schematic representation of an exemplary artificial straight septum designed and added to the anatomical model, according to some embodiments of the invention;

FIG. 11 is a schematic representation of two support structures designed according to the original anatomical geometry, according to some embodiments of the invention;

FIG. 12 are a schematic representations of defined pressure areas in the implant, according to some embodiments of the invention;

FIG. 13 is a schematic representation of exemplary areas imported (as files) to perform static analysis and topology optimization, according to some embodiments of the invention;

FIG. 14 is a schematic representation of an exemplary output of a topology optimization representing the most stable anatomical part of the patient's anatomical nose, which represents the constructive core area, according to some embodiments of the invention;

FIG. 15 is a schematic representation of exemplary differences in pore sizes, according to some embodiments of the invention;

FIG. 16 is a schematic representation of Voronoi Lattice designed based on individual points constructed in differentiate distance along the volume that represents the original nose volume, according to some embodiments of the invention;

FIG. 17 is a schematic representation of the support layers, according to some embodiments of the invention;

FIGS. 18a-b are a flowchart of an exemplary general method of generating an implant and uses thereof, according to some embodiments of the invention; and

FIGS. 19a-g are schematic representations of generation of an implant for femoral cartilage reconstructions based on the methods as disclosed in FIGS. 18a-b, according to some embodiments of the invention.

### DESCRIPTION OF SPECIFIC EMBODIMENTS OF THE INVENTION

The present invention, in some embodiments thereof, relates to 3D printed cartilage implants and, more particularly, but not exclusively, to 3D printed cartilage implants comprising both spheroids and single cells.

### Overview

An aspect of some embodiments of the invention relates to cartilage bioengineering using human expandable cells, optionally seeded with bio-ink, on a tailor-made bioresorbable 3D-printed scaffold, applicable to any organ. In some embodiments, the scaffold is stable and it is configured to maintain the original shape of the implant after implantation. In some embodiments, the scaffold is a polymeric scaffold configured to allow carrying spheroids of cells (referred hereinafter just as “spheroids”). In some embodiments, the scaffold is 3D-printed from an, optionally fast, degradable polymer materials configured to degrade in a time range of months rather than years. In some embodiments, a mold of a scaffold is 3D-printed using a computerized design and then the scaffold is generated from the mold. In some embodiments, the scaffold is printed using direct 3D printing techniques. In some embodiments, the mold is then melted away from the scaffold, the scaffold is optionally cut to refine its form and cells are then seeded on the scaffold for later implantation in a subject in need thereof. In some embodiments, the implant comprises a fast degradable polymer (for example Polydioxanone, which is also called PDS or PDO), which quickly degrades in the body and is completely reabsorbed, for example, in about six months. In some embodiments, the implant comprises two layers configured to preserve the original shape of the implant after transplantation during the critical period, which can last several weeks, in which edema and scarring processes exert pressure on the implant. In some embodiments, the bottom layer of the two layers is a support layer that provides additional support to preserve the shape of the implant. In some embodiments, the upper layer allows for the cells to mature into neo-cartilage tissue during the window of opportunity in which the cells are in optimal condition in terms of function and vitality. In some embodiments, the relatively fast kinetics of PDO degradation in combination with the two-layer approach potentially allows for a minimum synthetic (polymeric) component and a maximum biologic (spheroids, cells and ECM produced by them) component. In some embodiments, a potential advantage of the implant is that it enables the use of spheroids in a stable scaffold. In some embodiments, the upper layer comprises large macropores. It is known that large macropores usually cause scaffold instability, therefore the upper layer is supported by the bottom PDO layer until the engineered tissue matures and then the stability is achieved by the features of the neo-cartilage itself. In some embodiments, bio-ink is not used and cells are seeded directly into the scaffold.

An aspect of some embodiments of the invention relates to a combination of 3D printed cartilage implants with tissue engineering. In some embodiments, the implant comprises a unique scaffold design having a fast degradable polymer (for example Polydioxanone, which is also called PDS or PDO) in combination with poly(lactic-co-glycolic acid) (PLGA). In some embodiments, a potential advantage is that it potentially allows for the right balance for stability of the graft with minimum scaffold material that lasts long term, allowing for the cellular component to colonize the scaffold and be part of the structure quicker. In some embodiments, a mold of a scaffold is 3D-printed using a computerized design and then the scaffold is generated from the mold. In some embodiments, the scaffold is printed using direct 3D printing techniques. In some embodiments, the mold is then melted away from the scaffold, the scaffold is optionally cut to refine its form and cells are then seeded on the scaffold for later implantation in a subject in need thereof. In some embodiments, the scaffold contains micropores (smaller than about 100 microns) and macropores (from about 300 microns to about 1200 microns). In some embodiments, the bi-porosity property of the scaffold is configured to allow carrying both spheroids and single cells (single cells loaded on it, or single cells migrating outside the spheroids). In some embodiments, the spheroids are formed via chondrocyte cell isolation and expansion. In some embodiments, the scaffold and the spheroids are combined to generate the graft. In some embodiments, the implants are configured to allow in vivo cartilage maturation and physical resistance, which are essential during natural scar formation, following transplantation of the implant into the patient. In some embodiments, a potential advantage of the implant is that it allows the use of spheroids with scaffolds since, to this date, most spheroidal systems today are scaffold-free. In some embodiments, a potential advantage of using spheroids is that spheroids are building blocks for tissue engineering, compared to 2D cell systems, additionally, spheroids exhibit an enhanced regenerative capacity. In some embodiments, exemplary locations for implantation are one or more of larynx, nose, ribs, trachea, external ear, joints, disks and bone.

An aspect of some embodiments of the invention relates to an implant for reconstructing a full-scale human autologous bioengineered cartilage tissue. In some embodiments, the implant comprises a synthetic biodegradable/bioresorbable clinical-grade scaffold that allows tissue growth. In some embodiments, the implant is characterized by two parameters: (a) rapid degradation of the scaffold over a period of months and (b) 3-dimensional chondro-spheroids seeded on the scaffold have a high chondrogenic potency. In some embodiments, a potential advantage of having an implant with these two parameters is that it potentially provides the implant with the bioengineered construct requirements that allows to mimic the endogenous cartilage properties. In some embodiments, the implant allows two processes, scaffold degradation and tissue formation, to occur simultaneously. In some embodiments, the implant allows the quick replacement of the scaffold with the developing cartilage. In some embodiments, a potential advantage of the implant is that it potentially allows to provide stable mature/functional neo-cartilage, which optionally completes its maturation after transplantation, which provides better structure integrity compared to the currently accepted approaches that use long-term scaffolds loaded with two-dimensional adherent cultured cells. In some embodiments, a potential advantage of the implant is that it potentially avoids undesirable post-transplantation grafts deformations, arising because of scar formation and incomplete tissue maturation, which are expected to occur in the patient. In some embodiments, a potential advantage of the implant is that it potentially enables the production of un-deformed physical-pressures/forces resistant constructs preserving the original shape and structure of the bio-engineered implant over a long time.

A potential advantage of the implant as disclosed herein, comprising a cell-carrying scaffold with a dedicated support scaffold is that it allows the generation of a stable scaffold for an implant which allows the seeding of spheroids (either of the same size or of different sizes), without worrying about the stability of the scaffold once the implant has been implanted. This potentially allows using spheroids, which are a better source of cells for implantation in comparison with single cell implantation. Additionally, since stability is not an issue (due to the support scaffold), locations where high pressure is expected to be applied on the implant are provided with bigger porous which will house spheroids that will regenerate the tissues at the location of the implant. This concept goes against of what has been done until today.

In some embodiments, the implant is configured for the reconstruction of cartilage in one or more of the knee, the tibia, the femur and the patella. For example, reconstruction of any articular cartilage due to injury or defect.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Implant

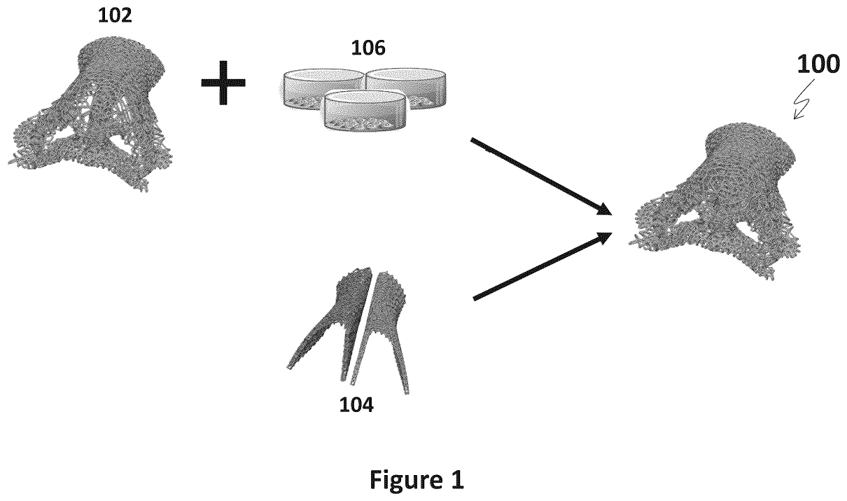

Referring now to FIG. 1, showing a schematic representation of an exemplary implant, according to some embodiments of the invention. In the following paragraphs, an exemplary implant for a nose will be used to explain the invention. It should be understood that implant directed to other locations in the body, for example one or more of larynx, ribs, trachea, disks, femoral bones, bones in general, locations requiring additions/modifications of tissue either for medical or cosmetic reasons, cartilage in one or more of the knee, the tibia, the femur and the patella, for example, reconstruction of any articular cartilage due to injury or defect external ear and joints, are also included in the scope of the invention and that the following explanations also covers those locations.

In some embodiments, the implant 100 is a personalized 3D printed cartilage implant with tissue engineering. In some embodiments, the implant is printed using direct 3D printing techniques. In some embodiments, the implant 100 comprises two layers 102/104, which are tailor made for the implant recipient. In some embodiments, both layers 102/104 are made from clinical-grade, biodegradable polymer material, for example, polydioxanone (PDO), which quickly degrades in the body and is quickly reabsorbed, for example in about six months. In some embodiments, additional materials that can be used are one or more of: poly(lactic-co-glycolic acid) (PLGA), poly l-lactide (PLA) and poly caprolactone (PCL). In some embodiments, the layers are made of different materials. In some embodiments, in an exemplary nose implant, the two layers are a nose-shaped layer 102 comprising a multiporous structure (see below), which serves as a scaffold for cell growth 106, and a support PDO layer 104, which provides mechanical stability to the nose-shaped layer 102 located above the support PDO layer 104. In some embodiments, the support PDO layer 104 comprises a form of the canal of the nose (nasal-canal form). In some embodiments, a mold of a scaffold is 3D-printed using a computerized design and then the scaffold is generated from the mold. In some embodiments, the mold is then melted away from the scaffold, the scaffold is optionally cut to refine its form and cells are then seeded on the scaffold for later implantation in a subject in need thereof.

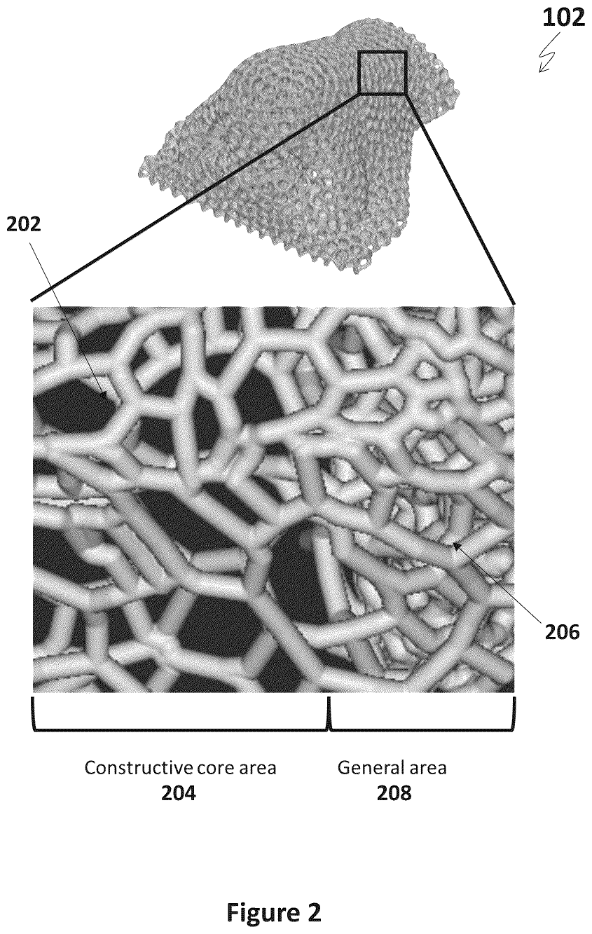

Referring now to FIG. 2, showing a schematic representation of an exemplary architecture of an exemplary nose-shape layer, according to some embodiments of the invention. In some embodiments, the nose-shape layer 102 comprises throughout its surface a combination of macropores 202, which generate what are herein referred to constructive core areas 204, and micropores 206, which generate what are herein referred to general areas 208. In some embodiments, the two areas comprise distinct mechanical characterizations. In some embodiments, the macropores 202 are smaller than about 100 microns. In some embodiments, the macropores 202 comprise a size from about 30 microns to about 70 microns. Optionally from about 20 microns to about 80 microns. Optionally from about 10 microns to about 100 microns. In some embodiments, the sizes of the micropores 206 are divided into three, partially overlapping, groups: 1) medium size macropores having a size from about 400 microns to about 600 microns; 2) large size macropores having a size from about 500 microns to about 900 microns; and 3) extra-large size macropores having a size from about 800 microns to about 1200 microns.

In some embodiments, the two distinct mechanical characteristic textures of the constructive core area and of the general area, as shown for example in FIG. 2, are dictated by the quantity of macropores and micropores in each area. In some embodiments, the constructive core area and the general area are design separately with a different pore size mix.

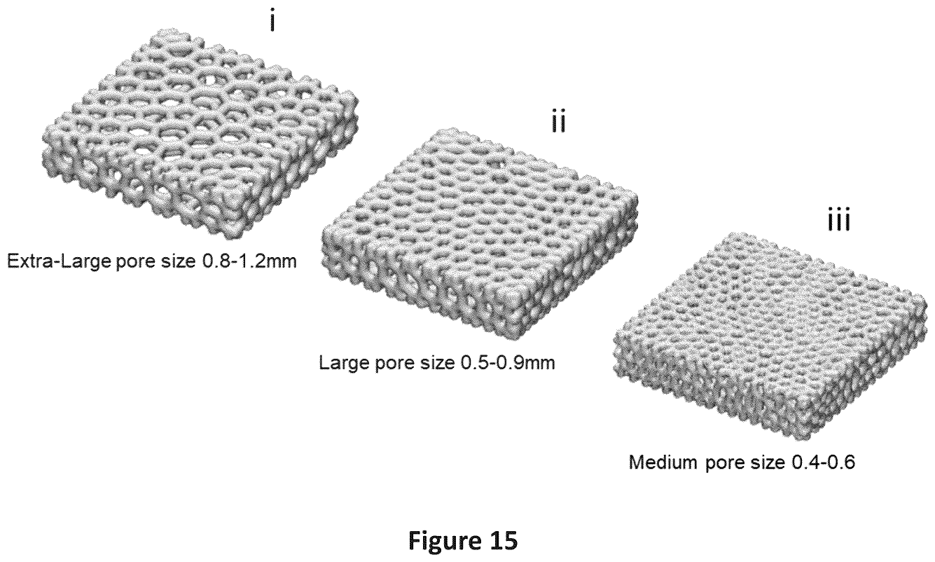

In some embodiments, the texture in the constructive core area contains:
From about 15% to about 30% extra-large pores having a size of from about 0.8 mm to about 1.2 mm (FIG. 15—ii);
From about 0% to about 30% large pores having a size of from about 0.5 mm to about 0.9 mm (FIG. 15—i); and
From about 40% to about 75% medium pores having a size of from about 0.4 mm to about 0.6 mm (FIG. 15—iii).

In some embodiments, the texture in the general area contains:
From about 5% to about 20% extra-large pores having a size of from about 0.8 mm to about 1.2 mm;
From about 5% to about 25% large pores having a size of from about 0.5 mm to about 0.9 mm; and
From about 55% to about 90% medium pores having a size of from about 0.4 mm to about 0.6 mm.

It is known in the art that there are locations in the implant that are subjected to different intensities of stress during the first months after implantation. For example, in the nose, there are mainly three areas that are subjected to more pressure than the others, the tip of the nose and the side areas above the alae (wings of the nose)—see also below for more explanations. In some embodiments, opposite to what has been seen until today, areas that are expected to suffer higher levels of pressure are going to be designed to comprise macropores. It should be emphasized that until today, the opposite has been done. It has been believed that areas that are expected to suffer higher levels of pressure should be generated to comprise micropores, which technically are stiffer. The inventors have surprisingly found that the opposite provide a better long term effect. Therefore, areas that are expected to suffer higher levels of pressure are going to be designed to comprise macropores. Additionally, those areas having macropores, which by nature are less stiff and therefore less stable, are supported by the support layer located below. Lastly, the inventors have found that by doing this, the neo-cartilage tissue allowed to grow in those areas of higher pressure comprising macropores, provide a better stable tissue, than tissue growth over micropores.

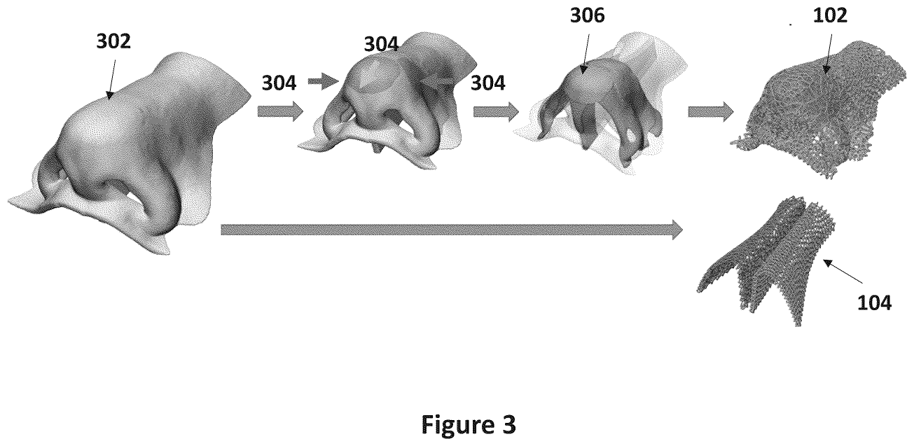

Referring now to FIG. 3, showing a schematic representation of an exemplary process of identification of areas under more mechanical stress, according to some embodiments of the invention. In some embodiments, as mentioned above, the pore-size composition of the nose-shape layer 102 is not uniform. For example, since specific areas 304 within the implant are under more mechanical stress than others, relevant areas 304 are individually determined using, for example, the patient's MRI/CT imaging 302, and those areas (as seen as a total area 306 within the implant) in the nose-shape layer 102 will be designed to contain a greater proportion of micropores 206, to accommodate for spheroid growth. In some embodiments, a potential advantage of having a scaffold comprising planned multi-porous properties is that it allows for both spheroids and single cells to optimally grow on the scaffold.

It is known in the art that spheroids require large pores, which have a downside of causing structural instability of the scaffolds, which is the reason why most spheroidal systems today are scaffold-free. In some embodiments, the support PDO layer 104 overcomes this challenge by providing stability to the nose-shape layer 102, in addition to the intrinsic stability provided to the scaffold. In some embodiments, the support PDO layer 104 comprises micropores having a size from about 400 microns to about 600 microns.

In some embodiments, a potential advantage of the two-layer approach is that it allows the bioengineered construct to mimic the endogenous cartilage properties and potentially provides the right balance between graft stability and minimal scaffolding. In some embodiments, the designed structure of the scaffold allows the cellular component to sufficiently colonize the scaffold, giving better long-lasting results.

### Exemplary Challenges Addressed by the System

A challenge of implants in general, is the ability to preserve the original required and desired shape after implantation, and specifically during the critical period of several weeks in which edema and scarring processes exert pressure on the implant. In some embodiments, the support PDO layer 104 provides additional support to preserve the original required and desired shape after implantation. Additionally, in some embodiments, by using the abovementioned two-layer approach, the nose-shape layer 102 allows for the cells to mature into neo-cartilage tissue during the window of opportunity in which the cells are in optimal condition in terms of function and vitality, while the support PDO layer 104 provides the strength needed to retain the original required and desired shape. In some embodiments, the two-layer approach allows for a minimum synthetic (polymeric) component and a maximum biologic (spheroids and cells) component. Lastly, as mentioned before, the implant enables and provides a stable scaffold for spheroids. As mentioned before, it is known that large macropores usually cause scaffold instability, but are also required for the use of spheroids. In some embodiments, the nose-shape layer 102 comprising the large macropores and the spheroids is supported by the support PDO layer 104 until the engineered tissue matures and the stability is achieved by the features of the neo-cartilage itself. In some embodiments, a potential advantage of the implant is that is potentially provides optimal conditions throughout the different phases of the implant process. For example, during the in-vitro stage, the different size pores support the growth of both individual cells and spheroids of different sizes, while during the transplantation phase, the support PDO layer 104 gives additional support in retaining the intended shape of the implant during the critical period when swelling and other forces might contort the shape of the implant, and lastly, the biological tissue is given the optimal conditions to continue to support the implant in the long term.

### Exemplary Overview of an Exemplary Process of Preparation of the Exemplary Implant

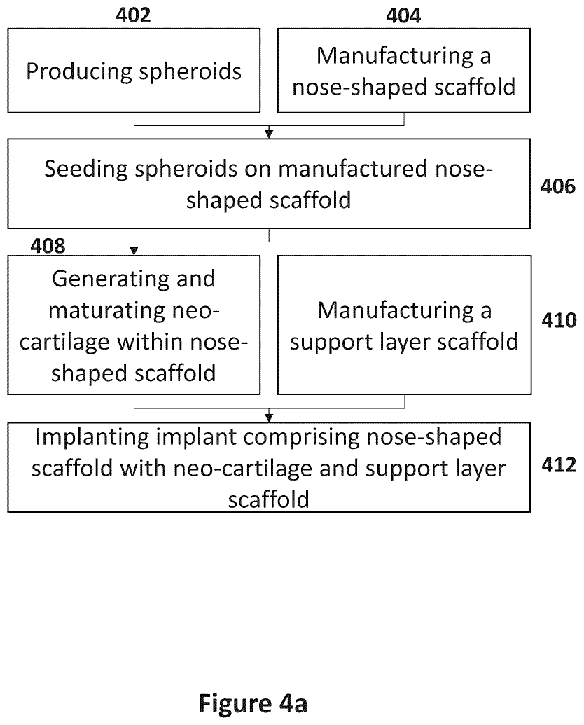

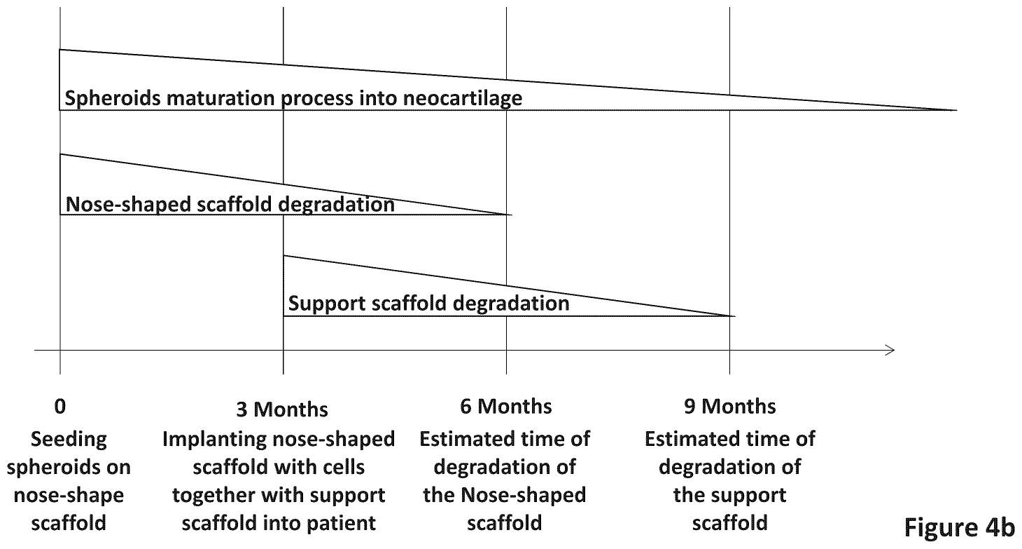

In some embodiments, a personalized shape of the two scaffolds of the implant are virtually generated. In some embodiments, the nose-shape layer 102 scaffold is printed first, while the support PDO layer 104 scaffold is printed later. In some embodiments, the nose-shape layer 102 scaffold is seeded with cells, for example, chondro-spheroids (and inherently also single cells), formed from expanded chondrocyte cells. In some embodiments, single cells also migrate out from the spheroids during the expansion period. In some embodiments, between about 1 month and about 3 months after performing the seeding and the in-vitro differentiation, the construct of the nose-shape layer 102 scaffold with cells is transplanted into the patient along with the support PDO layer 104. In some embodiments, the nose-shape layer 102 scaffold is expected to be completely degraded in the body within about 3 months and about 4 months. However, in some embodiments, the support PDO layer 104, that did not undergo the process of in-vitro differentiation prior to transplantation as the nose-shape layer 102 scaffold, will remain intact for additional from about 2 months to about 3 months, and continue to provide mechanical support until the full maturation of neo-cartilage (see below—FIG. 4b).

Referring now to FIG. 4a, showing a flowchart of an exemplary process of preparation of an exemplary implant, according to some embodiments of the invention. In some embodiments, the process comprises producing spheroids 402. In some embodiments, producing spheroids comprises isolating chondrocyte cells, expanding the cells and forming the spheroids (see below exemplary methods). In some embodiments, the cells are chondrocytes only. In some embodiments, the cells are a combination of chondrocytes and mesenchymal stem cells (MSCs).

In some embodiments, the process comprises manufacturing a nose-shaped scaffold 404. In some embodiments, manufacturing a nose-shaped scaffold 404 comprises designing and printing the nose-shaped scaffold. In some embodiments, a mold of a scaffold is 3D-printed using a computerized design and then the scaffold is generated from the mold. In some embodiments, the scaffold is printed using direct 3D printing techniques. In some embodiments, the mold is then melted away from the scaffold, the scaffold is optionally cut to refine its form and cells are then seeded on the scaffold for later implantation in a subject in need thereof. In some embodiments, as mentioned above, the scaffold is made of the polymer PDO. In some embodiments, a potential advantage of using PDO is that PDO is a fast degradable (bioresorbable) polymer (PDS), which additionally potentially provides the right balance of graft stability with minimum scaffold material that lasts the time necessary for the cellular component to colonize the scaffold and become part of the structure. In some embodiments, as mentioned above, the scaffold contains micropores (for example, smaller than 100 microns) and macropores (from about 400 microns to about 1200 microns). In some embodiments, this bi-porosity property of the scaffold is configured to allow carrying both spheroids and single cells (single cells loaded on it or single cells migrating out of the spheroids).

In some embodiments, producing the spheroids 402 and manufacturing a nose-shaped scaffold 404 are separate independent actions that are synchronized to have the nose-shaped scaffold ready once the spheroids arrive at the required growth level.

In some embodiments, the process comprises seeding the spheroids on the manufactured nose-shaped scaffold 406 (see below exemplary methods).

In some embodiments, the process comprises generating and maturating neocartilage within the nose-shaped scaffold 408 (see below exemplary methods).

In some embodiments, the process comprises manufacturing a support layer scaffold 410. As mentioned above, in some embodiments, a mold of the support layer scaffold is 3D-printed using a computerized design and then the support layer scaffold is generated from the mold. In some embodiments, the scaffold is printed using direct 3D printing techniques. In some embodiments, the mold is then melted away from the support layer scaffold, the support layer scaffold is optionally cut to refine its form.

In some embodiments, generating and maturating neocartilage within the nose-shaped scaffold 408 and manufacturing a support layer scaffold 410 are separate independent actions that are synchronized to have the support layer scaffold ready to be used once the nose-shaped scaffold with cells arrive at the required growth and maturation level.

In some embodiments, the process finishes by implanting the implant comprising the nose-shaped scaffold with the cells and the support layer below it, on a patient 412.

Referring now to FIG. 4b, showing a schematic representation of an exemplary timeline of implantation, according to some embodiments of the invention. In some embodiments, a potential advantage of the implant is that it combines two critical features: on one side it allows the growth of neocartilage tissue from spheroids; on the other side the predetermined and calculated timing of degradation of the scaffolds enable the optimal incorporation of the neocartilage into the body. This is because, on one side, the upper layer (containing the spheroids) degrades in the first three months after implantation, while the support layer, providing support to the neocartilage maturing on the upper layer, stays for at least 6 months after the moment of implantation. FIG. 4b schematically shows an exemplary timeline of the whole process. In some embodiments, at T0, the spheroids are seeded into the nose-shape layer 102 scaffold. In some embodiments, after 3 months, the nose-shape layer 102 scaffold with the cells is ready for implantation. In some embodiments, therefore, the support PDO layer 104 scaffold is printed and both of them, the nose-shape layer 102 scaffold with the cells together with the support PDO layer 104 scaffold, are implanted into the patient. In some embodiments, about 3 months after implantation, which are about 6 months from T0, it is estimated that the nose-shape layer 102 scaffold will completely degrade, while the support PDO layer 104 scaffold will stay for another about 3 months, which are about 9 months from T0. In some embodiments, this means that the support PDO layer 104 scaffold will practically provide support for a period of time of about 6 months after implantation.

### Exemplary Cells

In some embodiments, spheroids can be produced from different types of cells. In some embodiments, the source of the spheroids is stem cells, for example MSCs or iPS, which can then be sorted into a variety of tissues. For example, spheroids of mesenchymal stem cells can be sorted into cartilage, bone and fat. Therefore, in some embodiments, an implant can be produced of cartilage for example for nose; or an implant can be produced of bone for example for skull bones; or an implant can be produced of fat for example for breast reconstruction. In some embodiments, spheroids can be produced from cells isolated from a biopsy of muscle, bone, fat, lung epithelium, kidney epithelium, either from fetal or adult tissues. In some embodiments, spheroids can be produced from human cells as well as from other mammalian cells. It should be understood that the above-mentioned are examples only provided to allow a person having skills in the art to understand the invention and should not be limiting in any way.

In some embodiments, the multiporous scaffold described herein is configured to allow carrying one or more varieties of cells/spheroids, for example, a trachea can be produced by seeding the scaffold with cartilage cells on the outside surface of the scaffold and seeding epithelial cells on the inside surface of the scaffold. In some embodiments, optionally, cartilage is generated by seeding a combination of chondrocyte spheroids and MSCs spheroids.

A potential advantage of the scaffold with cells of the present invention is that it potentially allows the generation of any kind of multiporous scaffold (comprising a plurality of diverse sizes of porous) and seed therein the required type or multiple required types of cells and/or spheroids, which allows the production of a variety of tissues depending on the form (design) of the scaffold, the source of the cells and the differentiation medium.

### Exemplary General Methods

### Formation of Chondro-Spheroids

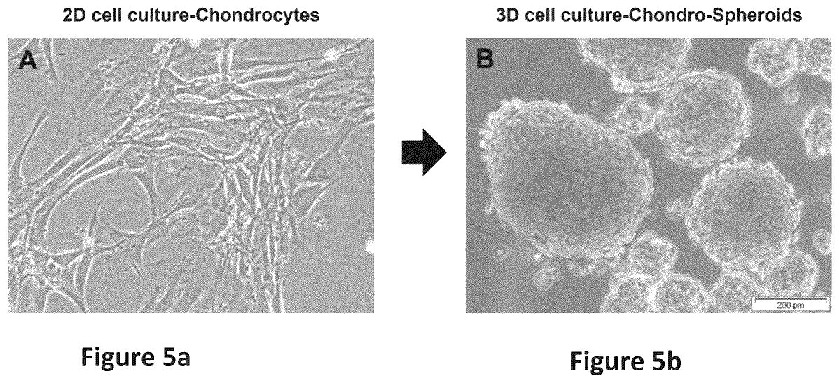

Chondro-Spheroids were formed from 2D-monolayer cultured chondrocytes. First, chondrocetes were expanded as a monolayer on tissue culture flasks in growth medium. After reaching confluence, the cells were detached and moved into flasks pre-coated with Poly(2-hydroxyethyl methacrylate) and facilitate spheroid formation. After 7 days in culture, spheroids between about 100 micron and about 1000 micron were formed. FIG. 5 shows light microscope images of the formation of chondro-spheroids. In FIG. 5a, “A” shows adherent monolayer chondrocytes in culture (2D cell culture), while in FIG. 5b, “B” shows spheroids formed from 2D cells (3D chondro-spheroids).

In Vitro Scaffold-Free Cartilage Formation of 2d Chondrocyte Culture Vs. Chondro-Spheroids

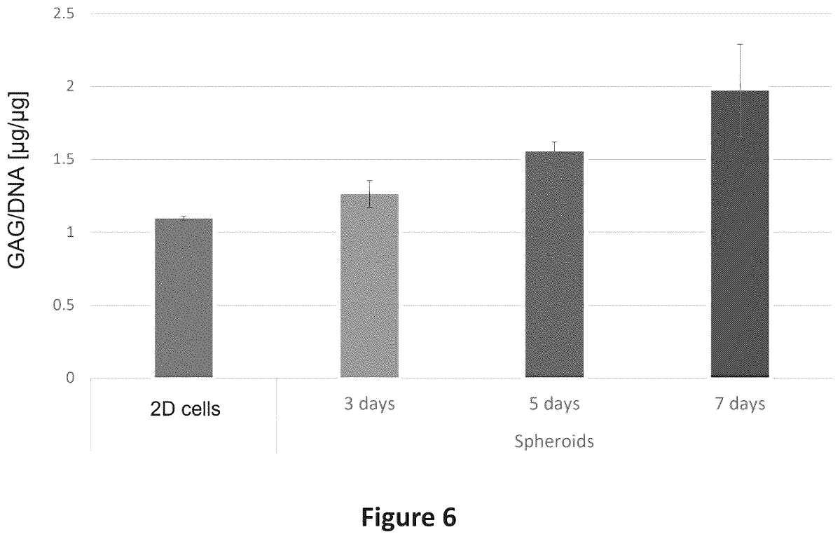

To assess the chondrogenic differentiation potential of 2D cells and chondro-spheroids, scaffold-free constructs (plugs) were created from chondrocytes grown as 2D cell culture or from 3D cultured chondro-spheroids grown for 3, 5 or 7 days. The plugs were grown in growth medium for additional 2 days, then differentiated in vitro for 3 weeks. Chondrogenic potential was assessed by measuring the levels of proteoglycan as sulfated glycosaminoglycan (GAG) content, normalized to DNA content, with spheroids grown for 7 days having the highest proteoglycan level. FIG. 6 shows the results of cartilage formation in scaffold-free chondrocytes in vitro in 2D cell culture vs. chondro-spheroids.

### Fibrin Bio-Ink Supports Chondrogenic Differentiation of Spheroids Into Functional Engineered Cartilage

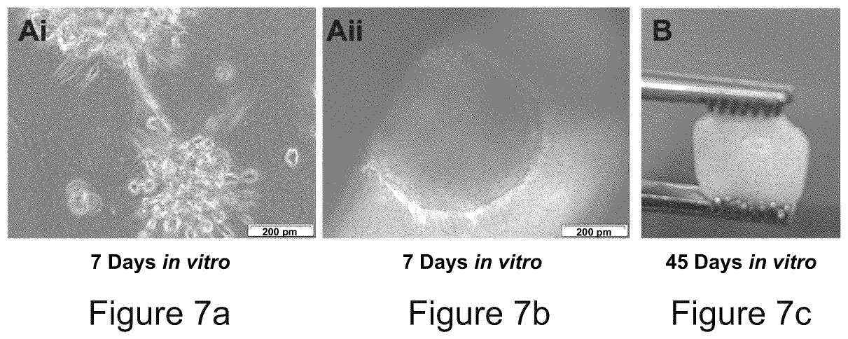

Chondro-spheroids were created as described above and cultured in vitro for 7 days. Afterwards, spheroids were collected and seeded in fibrin bio-ink and cultured for additional 45 days in vitro. In FIGS. 7a and 7b show representative microscope images of differentiation of spheroids. Single cells migrating out of the spheroids are visible. As seen in FIG. 7c, after 45 days in vitro, the spheroids have differentiated into cartilage-secreting cells that produced mature and functional engineered cartilage tissue, indicating their chondrogenic potential. In some embodiments, other materials can be used a bio-ink support, for example, bio-inks developed from hydrogels, biopolymer hydrogels that have been used for bioprinting including, but are not limited to, alginate, agarose, cellulose, collagen, fibrin, gelatin, gellan gum and hyaluronic acid. Alginate is a negatively charged polysaccharide derived from brown algae, and is one of the most commonly used hydrogels in both tissue engineering and bioprinting, gelatin methacrylol (GelMA), collagen, poly(ethylene glycol) (PEG), Pluronic®, alginate, and decellularized extracellular matrix (ECM)-based materials and amino-acids. In some embodiments, no bio-ink is used and/or necessary for the differentiation of the spheroids.

### In Vitro Maturation of Spheroids-Based PDO Scaffold-Based Engineered Cartilage

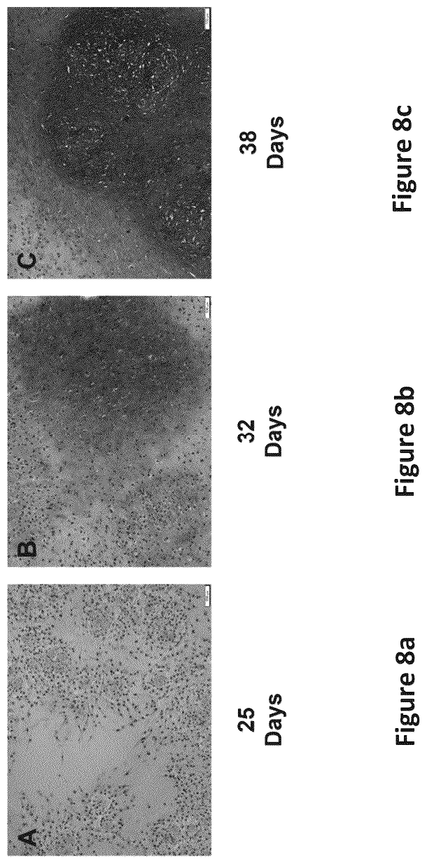

Chondro-Spheroids were suspended in fibrin bioink and seeded on a 3D-printed PDO scaffold. The construct was then incubated for 25 days, 32 days and 38 days in vitro. H&E histology analysis demonstrated formation and maturation of a functional engineered neocartilage over time. FIGS. 8a-c show exemplary in vitro maturation of spheroids-based PDO scaffold-based engineered cartilage. H&E staining of chondro-spheroid-PDO constructs incubated in vitro for FIG. 8a “A” 25 days, FIG. 8b “b” 32 days and FIG. 8c “C” 38 days.

### Subcutaneous Implantation of a Nose-Shaped Functional Engineered Cartilage

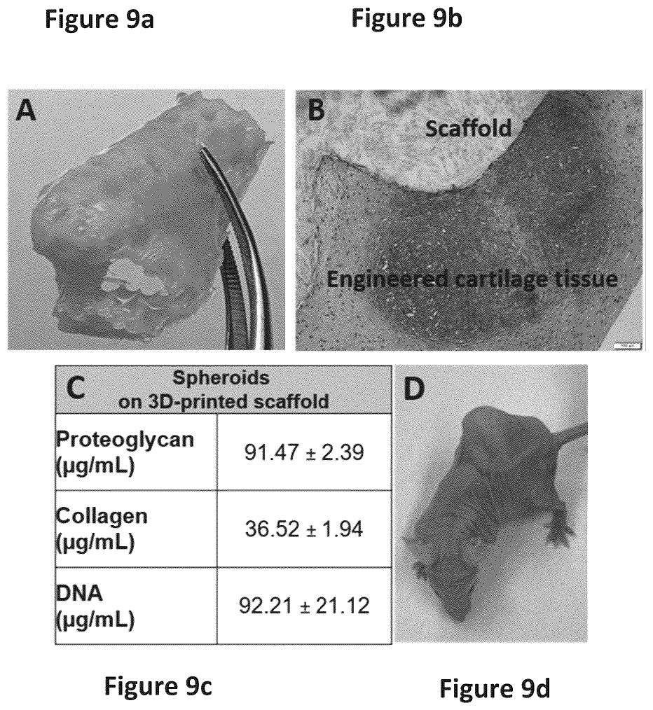

Chondro-spheroids were seeded in fibrin bio-ink onto 3D printed nose-shaped PDO scaffolds and cultured in vitro for 42 days, as shown for example in “A” in FIG. 9a. In some embodiments, other materials can be used a bio-ink support, for example, bio-inks developed from hydrogels, biopolymer hydrogels that have been used for bioprinting including, but are not limited to, alginate, agarose, cellulose, collagen, fibrin, gelatin, gellan gum and hyaluronic acid. Alginate is a negatively charged polysaccharide derived from brown algae, and is one of the most commonly used hydrogels in both tissue engineering and bioprinting, gelatin methacrylol (GelMA), collagen, poly(ethylene glycol) (PEG), Pluronic®, alginate, and decellularized extracellular matrix (ECM)-based materials and amino-acids. In some embodiments, no bio-ink is used and/or necessary for the differentiation of the spheroids. After in vitro differentiation, some constructs were fixed in 4% PFA, sectioned and stained with H&E, others were digested in papain and subjected to biochemical analysis. A third group of constructs were subcutaneously implanted into Athymic nude mice. As seen in FIGS. 9b-d, the spheroids formed a mature cartilage tissue with typical morphology, as shown by H&E staining in “B” in FIG. 9b, and high levels of collagen and proteoglycan, as expected from mature cartilage in “C” in FIG. 9c. The neo-cartilage tissue is expected to complete its'maturation process in vivo, as demonstrated in nude mice in “D” in FIG. 9d.

### Exemplary Implant Without Support Scaffold

In some embodiments, an implant does not require a support scaffold to perform as an implant. In some embodiments, the implant will comprise the same characteristics as the implant disclose above, meaning, an implant comprising two or more zones within the implant each having distinct sizes of porous. In some embodiments, the implantation zone does not require the implant to have a support scaffold in order to correctly perform. In some embodiments, the implantation zone provides the required support to the implant.

### Exemplary Locations for Implanting the Implant

In some embodiments, the implant of the present invention can be implanted in places where implants require secondary support scaffolds, like in the nose, and can be implanted in places where no other scaffolds are required, for example, implants used as disks, vertebrae, joints, femoral bones, bones in general, and in locations that require addition and/or changes of volume either for medical or cosmetic reasons. For example, cartilage in one or more of the knee, the tibia, the femur and the patella, for example for reconstruction of any articular cartilage due to injury or defect.

### Exemplary Additional Components Within the Implant

In some embodiments, the implant comprises one or more additional materials and/or components that are configured to be released from the implant once implanted, for example, the implant can comprise drugs, steroids, antibiotics, anticoagulants, and other.

### Exemplary Combinatorial Embodiments

Over the present invention several features were explained regarding the same or different embodiments of the invention. For example, in one embodiment, the implant comprises a bio-ink. In another embodiment, the implant is covered with the bio-ink. In another embodiment, the bio-ink is part of the materials that the implant are made of. In another embodiment, the implant comprises bio-ink as part of its materials and it is further covered with additional bio-ink. In some embodiments, the implant does not comprise bio-ink at all. In some embodiments, any of the abovementioned implants comprise cells and/or spheroids and/or single cells. In some embodiments, any of the abovementioned implant comprise one or more of releasable drugs, steroids, antibiotics and anticoagulants.

### Exemplary Materials and Methods

### Generation of a 3D Digital Model Based on the Patient-Specific Nose

A DICOM format (Digital Imaging and Communications in Medicine) CT scan of a nose was imported into the Mimics Software (Materialise) and was segmented to create a mesh model which afterwards exported as an unrefined 3D file in Stereolithography (STL) format.

### Artificial Geometry Addition and Manipulation of the Anatomical Model

The unrefined STL was processed in 3Matic Software (Materialise) to remesh and smooth the model. Thickness of 2 mm has been applied to the nose surface.

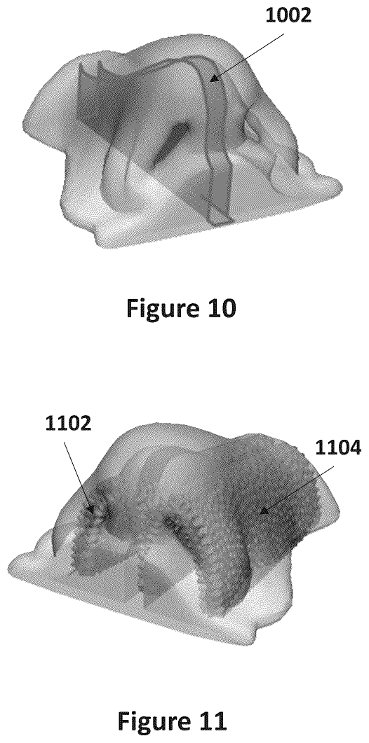

An artificial planar septum 1002 was designed and added to the anatomical model, as shown for example in FIG. 10, the surface thickness was 1.5 mm.

The support layer, which contains two tube structures 1102/1104, were designed according to the original anatomical geometry, as shown for example in FIG. 11, the surface thickness was 1.5 mm.

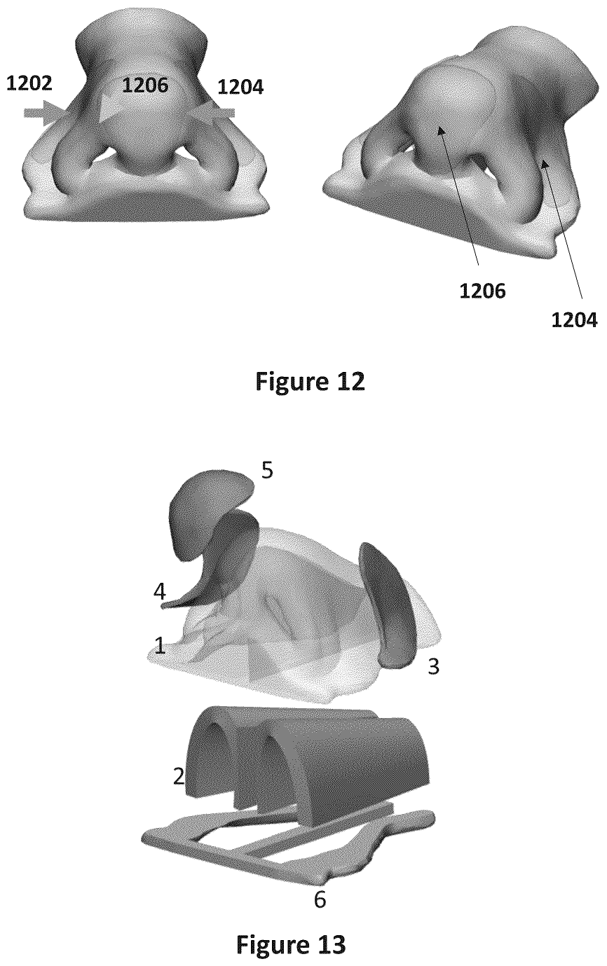

Definition and separation of 3 different zones of interest: pressure area left 1202, pressure area right 1204 and tip of the nose pressure area 1206, as shown for example in FIG. 12. The different areas represent the critical pressure area on the nose which are due to gravity compressive and tensile forces resulted by the natural healing process that includes edema and scarring.

### Extracting the Constructive Core Area by Topology Optimization

To perform Static analysis and topology optimization, the files were imported to the Ntopology software separately. The files are, as schematically shown in FIG. 13:
1. Original anatomical nose;
2. Structural inner tubes;
3. Pressure area left;
4. Pressure area right;
5. Tip of the nose pressure area; and
6. Fixed area that represents the connection area between the nose and the face.

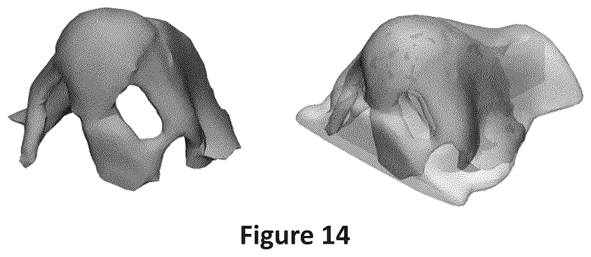

The output of the topology optimization was an implicit body that represents the most stable anatomical part of the patient's anatomical nose, as shown for example in FIG. 14. By the implicit body geometry, the nose model has been divided to two separate parts.

### Designing a Lattice Possess Two Areas of Different Multiple Porous Composition

Two lattice textures for the constructive core area and for the general area, as shown for example in FIG. 2, were design separately with a different pore size mix.

The texture in the constructive core area contains: 15-30% extra-large pore size (0.8-1.2 mm, FIG. 15—ii), 10-30% large pore size (0.5-0.9 mm, FIG. 15—i) and 40-75% medium pore size (0.4-0.6, FIG. 15—iii).

The texture in the general area contains: 5-20% extra-large pore size (0.8-1.2 mm), 5-25% large pore size (0.5-0.9 mm) and 55-90% medium pore size (0.4-0.6).

Hence the nose-shaped layer (FIG. 2) possesses a lattice with 2 compositions, the lattice of the constructive core area contains at least 3 times more extra-large pores and at least 2 times larger pores than the general area lattice.

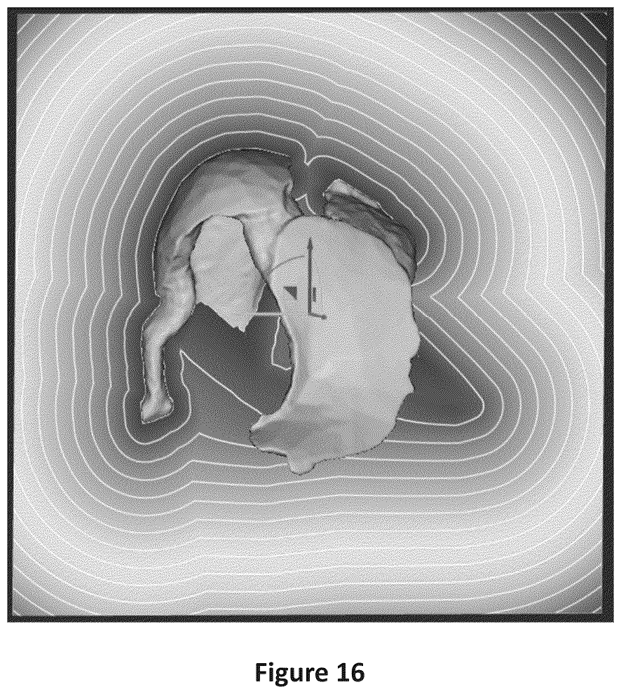

Voronoi Lattice were designed based on individual points constructed in differentiate distance along the volume that represents the original nose volume. The deviation of the points in the volume and the distance between each point to another ramped based on the distance of each point to the nose's core part, as shown for example in FIG. 16.

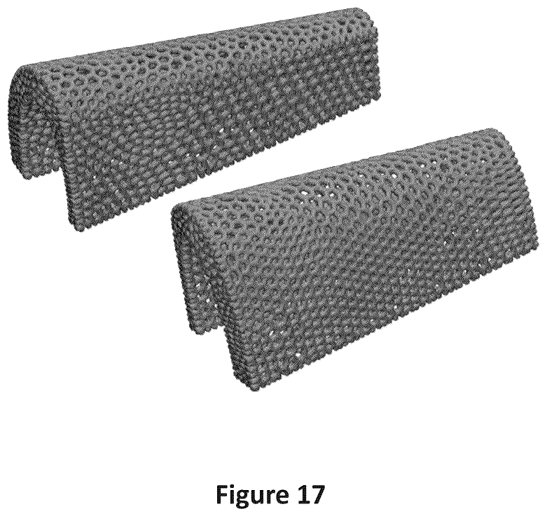

For Support layers, Voronoi Lattice were designed with a fixed pore size of 0.4-0.6 mm, as schematically shown in FIG. 17.

Thicken of 2 mm and 1.5 mm been applied to the lattice beams of the nose-shaped layer and the support layer respectably. The files were meshed to stl file format and exported to print in Prusa 3D printer.

### Fabricating the Scaffold by FDM 3D Printer

The scaffold and the water-soluble supporting box printed from Polydioxanone/PDO (Lattice medical) and BVOH (Verbatim) 1.75 mm filaments respectively. The Slic3r Prusa slicing software was used to plan the printing path: thickness of each layer 0.2-0.4 mm, printing speed 20-60 mm/s, extrusion temperature 170-210° C., build plate temperature 60-80° C. The files were then saved in g-code and imported to a Prusa MK3.1 printer with a 0.2/0.4 mm nozzle for 3D printing. In some embodiments, a mold of a scaffold is 3D-printed using a computerized design and then the scaffold is generated from the mold. In some embodiments, the mold is then melted away from the scaffold, the scaffold is optionally cut to refine its form and cells are then seeded on the scaffold for later implantation in a subject in need thereof. In some embodiments, the scaffold is printed using direct 3D printing techniques.

### Cell Culture

### Cell Isolation and Expansion

Tissue samples were collected according to the principles expressed in the Declaration of Helsinki and was approved by the Institutional Review Boards of Sheba Medical Center.

Monolayer 2D chondrocytes cell culture: chondrocytes were isolated from either costal cartilage or nasal cartilage and cut into 1-3 mm pieces, and incubated with collagenase II for 12-14 hours. The tissue solution was then filtered through a 100 μm strainer, washed with Growth medium (40 ml of DMEM F12 with 10% FBS and 1% Pen strep), and centrifuged at 600×g for 8 minutes. Cells were mixed with growth medium and seeded on T-175 flasks. Cells derived from ˜100 mg tissue were seeded per flask. Growth medium was changed every 2-3 days. Upon reaching confluence of 80%-100%, the cells were harvested and frozen in NutriFreez cryopreservation medium (Biological industries). Chondrocytes were thawed and grown as 2D monolayers for 2 passages.

Chondro-spheroid cell cultures: chondrocytes at passage 2 were harvested and seeded on poly (2-hydroxyethylmethacrylate) (poly-HEMA; Sigma-Aldrich)-precoated flasks, in NS growth medium, at a concentration of 5-15×104 cells/mL, allowing spontaneous formation of spheroids.

### Cell Seeding

Before seeding, PDO scaffolds were sterilized with 70% ethanol and U.V. eradiation, washed three times in PBS and soaked in growth medium.

Chondro-spheroids were seeded in Tisseel fibrin sealant (Baxter) to mediate cell attachment to the scaffold. Chondro-spheroids were collected, washed with PBS and re-suspended in thrombin solution (5 U/mL), then fibrinogen (45 mg/mL) was added, and the spheroids were quickly seeded onto the scaffold. For a 40% size nose shaped scaffold ˜100×106 cells in 350 μL fibrin solution were seeded. For 1 cm size disc-shaped PDO scaffold ˜7×106 cells in 25 μL fibrin solution were seeded. In some embodiments, other materials can be used a bio-ink support, for example, bio-inks developed from hydrogels, biopolymer hydrogels that have been used for bioprinting including, but are not limited to, alginate, agarose, cellulose, collagen, fibrin, gelatin, gellan gum and hyaluronic acid. Alginate is a negatively charged polysaccharide derived from brown algae, and is one of the most commonly used hydrogels in both tissue engineering and bioprinting, gelatin methacrylol (GelMA), collagen, poly(ethylene glycol) (PEG), Pluronic®, alginate, and decellularized extracellular matrix (ECM)-based materials and amino-acids. In some embodiments, no bio-ink is used and/or necessary for the differentiation of the spheroids.

### In-Vitro Differentiation

Seeded constructs were incubated for 1 hour at 37° C., followed by the addition of NS growth medium. 2-3 days after seeding, differentiation medium was added: DMEM F12 supplemented with pen-strep (1%, Biological Industries), TGF-β (10 ng/ml, Prospec), ITS premix (50 mg/mL, Sigma), ascorbic acid (50 μg/mL, Sigma), dexamethasone (100 nM, Sigma), and amphotericin B (0.25 μg/mL, Biological Industries). The medium was changed every 2-3 days for 4 weeks.

After 4 weeks of differentiation, constructs were fixed with 4% Paraformaldehyde for histology analysis by H&E, alcian blue and safranin-O staining, or digested with papain solution for biochemical analysis, or implanted into mice for in-vivo experiments.

### Graft Implantation

The animal study was approved by the committee on the ethics of animal experiments of the Sheba. Athymic nude mice (male, 7-9 weeks old; Envigo) were anesthetized with isofluorane. Nose-shaped constructs were implanted subcutaneously through small incisions in the skin which were then sutured with 5-0 absorbable sutures. Mice were sacrificed after 12 weeks, and the grafts were extracted and subjected to mechanical testing, staining and biochemical analysis.

### Biochemical Assays

Samples were digested with papain solution (40 μg/mL in 20 nM ammonium acetate, 1 mM EDTA, and 2 mM dithiothreitol) for 48 hours at 65° C. DNA content was measured using the Hoechst dye-binding assay. Proteoglycan amount was quantified by measuring the amount of sulfated GAG using the 1,9-dimethylmethylene blue (DMMB) dye binding assay. Collagen content was quantified by hydrolyzing samples in HCl at 110° C. for 18hours, and then measuring hydroxyproline levels using the chloramine T/Ehrlich's spectrophotometric assay.

### Additional Examples of Tissue Reconstruction

In some embodiments, as mentioned above, tissue reconstruction can be performed in different locations (bones, nose, cartilage, face, etc.) and for different reasons (medical or cosmetic).

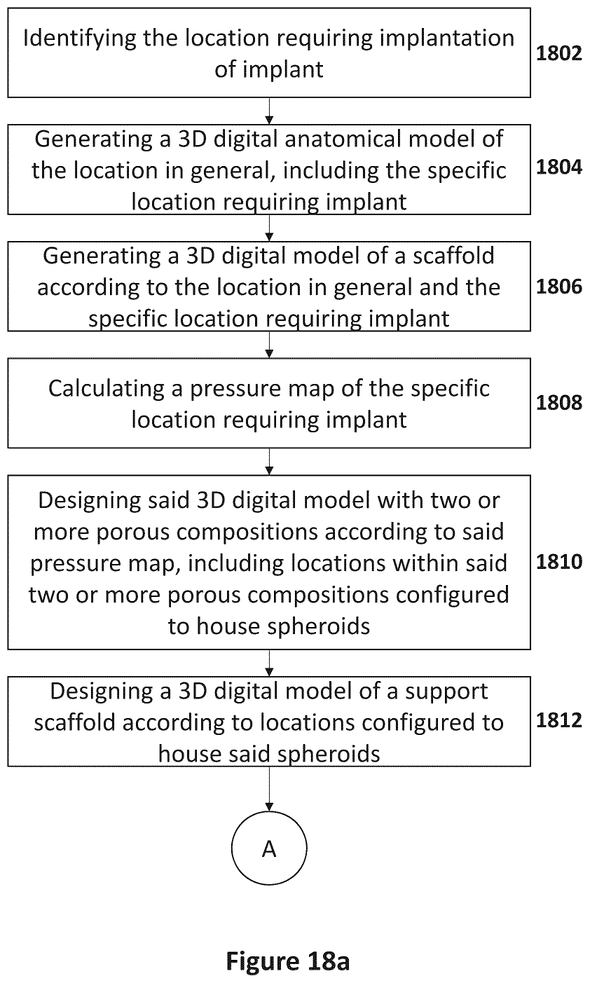

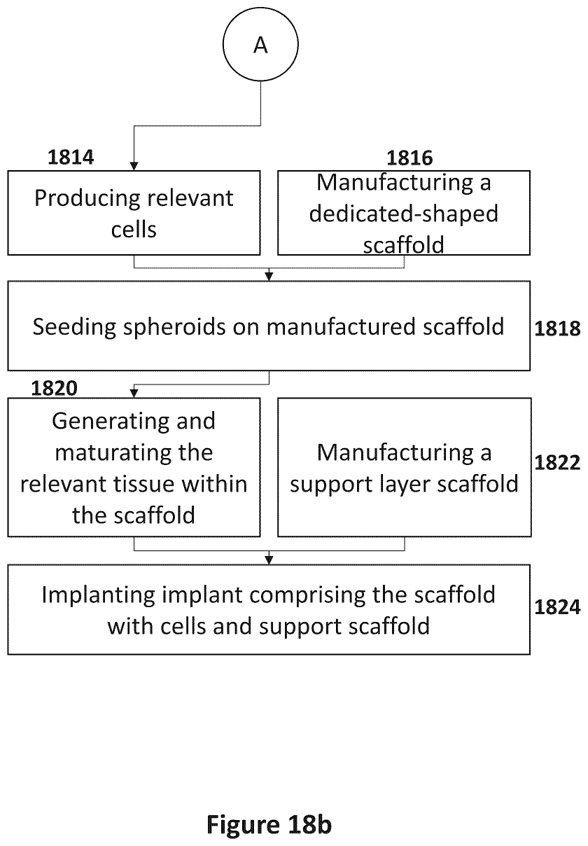

Referring now to FIGS. 18a-b, showing a flowchart of an exemplary general method of generating an implant and uses thereof, according to some embodiments of the invention.

In some embodiments, when generating an implant, a method comprises one or more of the following actions:

In some embodiments, a location requiring implantation is identified 1802.

In some embodiments, a 3D digital anatomical model is generated 1804. In some embodiments, the 3D digital anatomical model comprises the specific location requiring the implant and optionally also the surrounding areas adjacent to the specific location. In some embodiments, the 3D digital anatomical model is generated using one or more images, for example, CT images, MRI images, X-ray images, etc.

In some embodiments, a 3D digital model of a scaffold is generated 1806 according to the data generated before, and including the specific area requiring the implant. In some embodiments, at this stage, the 3D digital model is a generic model of the scaffold defining the general dimensions/form of the implant.

In some embodiments, a pressure map and/or a forces map of the specific location requiring an implant is generated 1808. In some embodiments, the pressure map/forces map is generated using known method is arts or it is provided by a priori using already known pressure data. In some embodiments, the pressure/forces map may include calculation of pressures/forces in different conditions, for example, dynamic or static pressures/forces applied on the implant.

In some embodiments, the 3D digital model of a scaffold is amended and/or further designed with two or more porous compositions according to the pressure map 1810. For example, zones of higher pressure will be provided with bigger pores than zones with lower pressure, as explained above. In some embodiments, designing the two or more porous compositions comprises including locations within the two or more porous compositions to be configured to house spheroids. In some embodiments, when designing the two or more porous compositions different conditions are taken under consideration, for example dynamic or static pressures/forces applied on the implant according to the specific location of the implant. For example, in the case of cartilage in the knee, movement of the knee causes a shift in the pressures/forces applied on the implant. In some embodiments, when designing the two or more porous compositions, the position of the compositions take under consideration the locations where the pressure/forces are applied on the implant so as to allocate one or the other.

In some embodiments, a 3D digital model of a support scaffold is designed 1812. In some embodiments, the support scaffold is designed to provide support to the areas that will house the spheroids and/or the areas comprising the bigger size pores.

Flowchart continues following the letter “A”to FIG. 18b.

In some embodiments, the process continues by producing the spheroids of the relevant type of cells 1814 (similar to what is disclosed herein elsewhere).

In some embodiments, the process continues by manufacturing the scaffold of the implant as designed in 1810 (1814).

In some embodiments, producing the spheroids 1814 and manufacturing a scaffold 1816 are separate independent actions that are synchronized to have the scaffold ready once the spheroids arrive at the required growth level.

In some embodiments, the process continues by seeding the spheroids on the manufactured scaffold 1818.

In some embodiments, the process continues by generating and maturating the relevant tissue within the scaffold 1820 (see below exemplary methods).

In some embodiments, the process comprises manufacturing the support scaffold as designed in 1812 (1822).

In some embodiments, generating and maturating the relevant tissue within the scaffold 1820 and manufacturing a support layer scaffold 1822 are separate independent actions that are synchronized to have the support scaffold ready to be used once the scaffold with cells arrive at the required growth and maturation level.

In some embodiments, the process finishes by implanting the implant comprising the scaffold with the cells and the support scaffold, on a patient 1824.

In some embodiments, as mentioned above, implants can be generated for different types of locations using the same principles of the methods as shown above and specifically as shown in FIG. 18a-b.

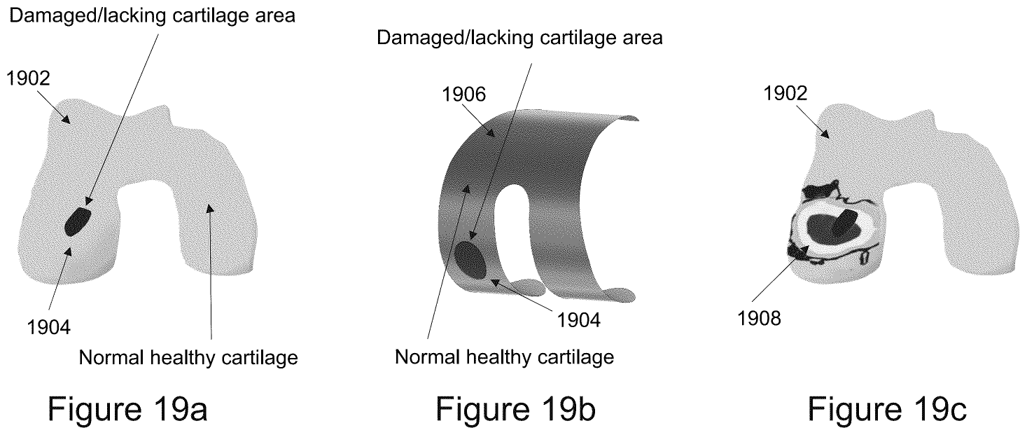

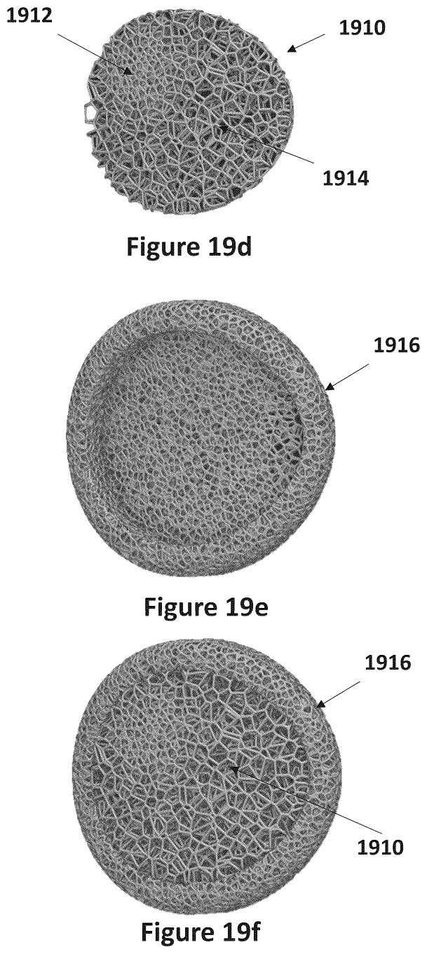

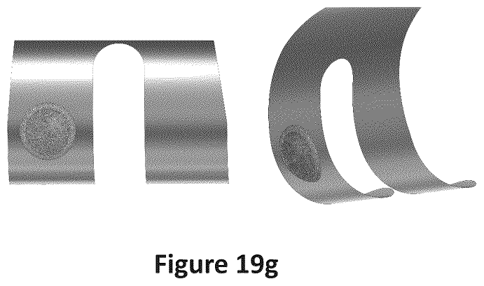

Referring now to FIGS. 19a-g, showing schematic representation of generation of an implant for knee cartilage reconstructions based on the methods as disclosed in FIGS. 18a-b, according to some embodiments of the invention.

FIG. 19a shows a scan of a knee area 1902, where cartilage needs to be reconstructed, schematically showing the specific location requiring an implant 1904. In this example, the area around the specific location 1904 is healthy tissue.

FIG. 19b shows a schematic 3D digital anatomical model of the location in general, including the specific location requiring implant (see 1804 in FIG. 18a).

FIG. 19c shows the generation/calculation of a pressure/forces map 1908 (see 1808 in FIG. 18a).

FIG. 19d schematically shows the scaffold of the implant 1910 having two or more porous compositions, for example smaller porous 1912 and bigger porous 1914 (“smaller” and “bigger” can be related to each other and/or as explained above in relation to the sizes)—(see design of scaffold in 1810 in FIG. 18a and its manufacturing in 1816 in FIG. 18b).

FIG. 19e schematically shows the support scaffold 1916 for the implant scaffold 1910 having a uniform porous composition (see design of support scaffold in 1812 in FIG. 18a and its manufacturing in 1822 in FIG. 18b).

FIG. 19f schematically shows the implant scaffold 1910 with the support scaffold 1916. In this example, the implant scaffold 1910 is located within the borders of the support scaffold 1916.

FIG. 19g is a schematic representation of the complete implant as shown over the 3D digital anatomical model of the location in general, including the specific location requiring implant.

In some embodiments, another example of generating an implant using the methods as disclosed above, is the generation of an implant for a bone in general, for example for the middle of a bone. In this example, the cells will generate bone tissue and not cartilage. In some embodiments, as mentioned above, any kind of tissues can be made into spheroids and implanted (grown) in the implant scaffold. Additionally, in this example, the organ comprises a cylindrical shape that is exposed to centripetal pressures/forces (centripetally high pressure/forces from the outside, and low pressure/forces from the inside), therefore, in this specific example, the scaffold containing cells is planned so the outer side will comprise the larger pores (constructive core area) and the inner part comprises smaller pores (general area). Lastly, in this example, the cell-carrying scaffold is implanted together with a support scaffold that wraps around the cell-carrying scaffold from all directions. This is different from the abovementioned examples where, for example, for the nose and for the knee, the cell-carrying scaffold is partially supported.

As used herein with reference to quantity or value, the term “about” means “within ±20% of”.

The terms “comprises”, “comprising”, “includes”, “including”, “has”, “having” and their conjugates mean “including but not limited to”.

The term “consisting of”means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An implant comprising:
a first scaffold comprising at least one first area characterized by micropores and at least one second area characterized by macropores, wherein said at least one second area is defined by being expected to be exposed to higher pressures and/or forces when compared with said at least one first area, and
at least one second scaffold configured to provide mechanical support to said first scaffold, wherein said first scaffold, and wherein said at least one second scaffold are configured to degrade at different time windows.

2-51. (canceled)

52. The implant according to claim 1, further comprising at least one bio-ink material configured to allow attachment of a plurality of cells to a surface of said implant, said bio-ink material is one or more of fibrin, hydrogel and amino-acids.

53. The implant according to claim 1, wherein said implant comprises a plurality of spheroids.

54. The implant according to claim 53, wherein said plurality of spheroids are chondro-spheroids.

55. The implant according to claim 53, wherein said macropores are configured in size for housing said plurality of spheroids.

56. The implant according to claim 53, wherein said plurality of spheroids are formed from one or more of expanded chondrocyte cells and mesenchymal stem cells.

57. The implant according to claim 53, wherein the implant is configured to gain stability from neo-cartilage tissue formed from said plurality of spheroids.

58. The implant according to claim 1, wherein said at least one first area comprises
a. from about 15% to about 30% extra-large pores having a size of from about 0.8 mm to about 1.2 mm;
b. from about 0% to about 30% large pores having a size of from about 0.5 mm to about 0.9 mm; and
c. from about 40% to about 75% medium pores having a size of from about 0.4 mm to about 0.6 mm.

59. The implant according to claim 1, wherein said at least one second area comprises
a. from about 5% to about 20% extra-large pores having a size of from about 0.8 mm to about 1.2 mm;
b. from about 5% to about 25% large pores having a size of from about 0.5 mm to about 0.9 mm; and
c. from about 55% to about 90% medium pores having a size of from about 0.4 mm to about 0.6 mm.

60. The implant according to claim 1, wherein said at least one second scaffold comprises a nasal-canal form.

61. The implant according to claim 1, wherein said at least one second scaffold is configured to degrade about 6 months after implantation.

62. The implant according to claim 1, wherein at least one second scaffold comprises polydioxanone.

63. The implant according to claim 1, wherein said first scaffold and said at least one second scaffold are made of different materials.

64. The implant according to claim 1, further comprising one or more of drugs, antibiotics, steroids and anticoagulants configured to be released from said implant after implantation.

65. A method of manufacturing an implant, comprising:
(a) printing a scaffold comprising at least one first area characterized by micropores and at least one second area characterized by macropores; and
(b) configuring said macropores so as to allow seeding spheroids therein.

66. The method according to claim 65, further comprising seeding said spheroids on said scaffold.

67. The method according to claim 65, wherein:
a. said printing said scaffold comprises printing said scaffold with at least one first area and at least one second area;
b. said at least one first area comprises
i. from about 15% to about 30% extra-large pores having a size of from about 0.8 mm to about 1.2 mm;
ii. from about 0% to about 30% large pores having a size of from about 0.5 mm to about 0.9 mm; and
iii. from about 40% to about 75% medium pores having a size of from about 0.4 mm to about 0.6 mm.
c. said at least one second area comprises one or more of medium size macropores, large size macropores and extra-large macropores; and
d. said at least one second area comprises:
iv. from about 5% to about 20% extra-large pores having a size of from about 0.8 mm to about 1.2 mm;
v. from about 5% to about 25% large pores having a size of from about 0.5 mm to about 0.9 mm; and
vi. from about 55% to about 90% medium pores having a size of from about 0.4 mm to about 0.6 mm.

68. The method according to claim 65,
further comprising printing at least one second scaffold, said printing said at least one second scaffold comprising providing said at least one second scaffold with a form so as to provide mechanical support to said scaffold,
wherein said printing said at least one second scaffold comprises one or more of:
a. printing said at least one second scaffold with a form of an internal surface of a location where said implant is needed to be implanted; and
b. printing said at least one second scaffold with a nasal-canal form.

69. The method according to claim 68, further comprising configuring said at least one second scaffold to degrade about 6 months after implantation.

70. An implant, comprising:
a. a first scaffold comprising a plurality of spheroids;
b. at least one second scaffold located below said first scaffold, and configured to provide mechanical support to said first scaffold.
